(19) United States
(12) Patent Application Publication
(10) Pub. No.: US 2007/0027710 A1
(43) Pub. Date: Feb. 1, 2007

(54) Method and system for automatically processing and evaluating medical data

(76) Inventor: Volker Mohr, Dusseldorf (DE)

Correspondence Address: GUDRUN E. HUCKETT DRAUDT, LONSSTR. 53, WUPPERTAL, 42289, DE

(21) Appl. No.: 10/552,729

(22) PCT Filed: Apr. 8, 2004
(86) PCT No.: PCT/DE04/00756

§ 371 Date: Jun. 30, 2006

(30) Foreign Application Priority Data
Apr. 8, 2003 (DE) 10316298.4

(51) Int. Cl. G06Q 10/00; G06F 19/00; G06Q 50/00; A61B 5/00

(52) U.S. Cl. 705/2; 705/3

(57) ABSTRACT

In a method for automatically processing and evaluating medical data for the purpose of determining and providing diagnostic and/or medical-care relevant information based on a plurality of available data sources, all data sources are acquired in a common data pool and each data source is evaluated based on predefined criteria. The results of the step of evaluating are saved in correlation to the data sources, respectively. The data contained in the data pool are referenced such that elements of the data pool that are correlated with regard to contents are linked and displayable automatically as being correlated. Based on the data sources in accordance with predetermined boundary conditions, a structured summary in the form of a virtual data sheet is produced.

### TECHNICAL FIELD OF THE INVENTION

The invention relates to a method and an arrangement for automatically processing and evaluating medical data for the purpose of determining and providing diagnostic and/or medical care-relevant information based on a plurality of available data sources.

In this context, the term “data source” is to be understood as all types of media that contain information in regard to a patient, i.e., x-ray images, computer tomography images, ultrasound images, blood tests, genetic analyses but also physician's reports and formal documents such as vaccination cards, prescriptions, medical opinions, and the like. Data sources can be present as an original, as a copy, in physical or electronic form.

### BACKGROUND OF THE INVENTION

Methods for automatic determination of diagnostically relevant parameters based on data of a precisely predetermined type, i.e., for automatic evaluation of medical section scan series as they are delivered by computer tomography or magnetoresonance tomography, are known.

DE 101 28 293 A1 discloses a method for determining a diagnostically relevant parameter based on electro-cardiographic and magneto-cardiographic data in which, based on the data, certain vectors are calculated and set into relation to one another in order to obtain a result with regard to certain heart diseases. An automatic processing and evaluation of data of different types in the sense of the present invention is not possible with the known methods.

WO 02/33654 A1 discloses a method and a corresponding arrangement for performing the method designed to support a physician in a concrete treatment situation such that the physician enters all the information that is currently available to him into a corresponding computer system that then provides concrete treatment proposals as a so-called “expert system”. However, the quality of the information that is available cannot be checked and evaluated by the expert system.

### SUMMARY OF THE INVENTION

The increasing possibilities in regard to information, particularly by means of the modern information technology, create the problem, not only in the field of medicine and medical care, that for a certain problem or, in the field of patient care, in regard to a certain patient, a lot of information is available that, on the one hand, must be considered for reasons of diligent care but whose significance often can be recognized only with difficulty.

Based on this, it is an object of the invention to provide a method and an arrangement with which data that originate from a plurality of different data sources can be processed and evaluated in accordance with uniform criteria in order to provide a decision-making aid to physicians, nursing personnel, and other members of the medical care professions and medical assistance professions in the case of diagnoses or therapeutic measures and similar decisions.

The object is solved by the method according to claim 1. Advantageous embodiments and realizations are disclosed in the dependent claims. The independent claim 10 concerns a corresponding arrangement for performing the method.

Further details and advantages of the invention result from the following description in connection with the drawing, wherein the description is to be understood to be purely exemplary and non-limiting.

### BRIEF DESCRIPTION OF THE DRAWINGS

FIG. 1 shows a schematic flowchart of the basic course of the method according to the invention.

FIG. 2 shows a detailed flowchart of the method step of evaluating a data source.

FIG. 3 shows a detailed flowchart of the method step of saving a data source.

FIG. 4 illustrates a possible form of representation of a virtual data sheet generated by the method according to the invention, which data sheet can be represented, for example, on a screen.

FIG. 5 shows a data sheet according to FIG. 4 with exemplary data.

FIG. 6 shows a detailed flowchart of the method step of referencing the data base.

FIG. 7 shows a principal schematic for illustrating the function of referencing.

FIG. 8 shows a detailed flowchart of the method step of validating the database.

FIG. 9 shows a schematic principal illustration of a possible arrangement for performing the method according to the invention.

FIG. 10 shows a first example of the course of the method step of processing a data source wherein the data source is an x-ray image.

FIG. 11 shows a second example of the course of the method steps of processing a data source wherein the data source is a physician's letter.

### DESCRIPTION OF PREFERRED EMBODIMENTS

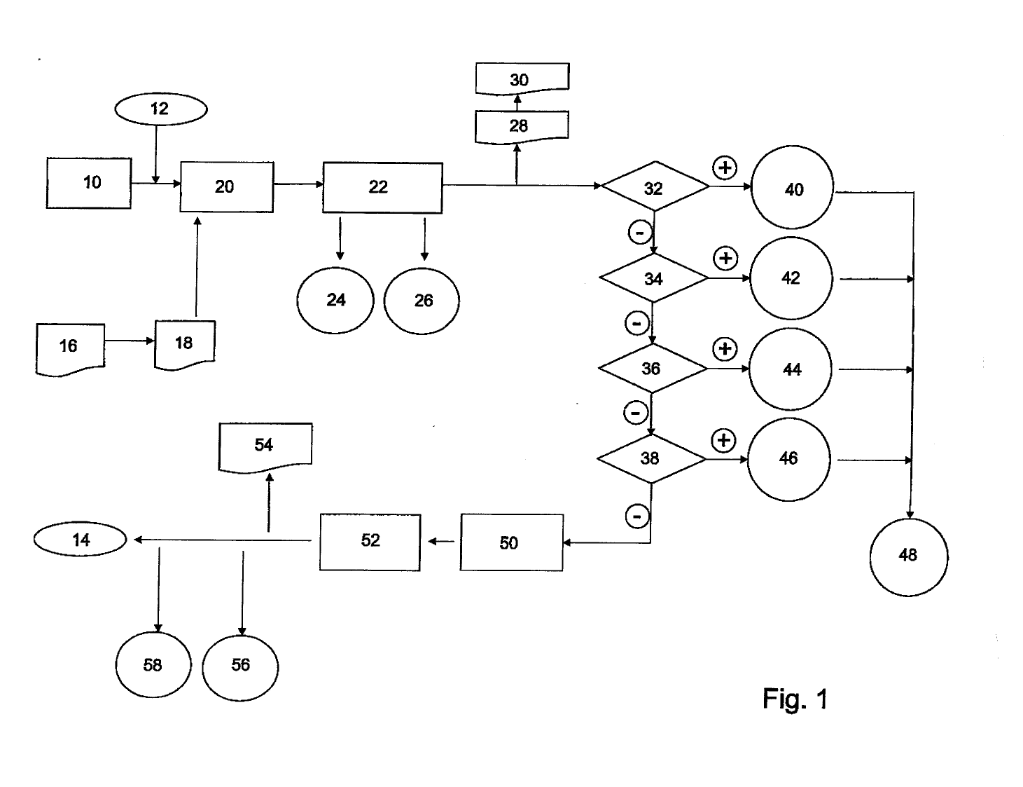

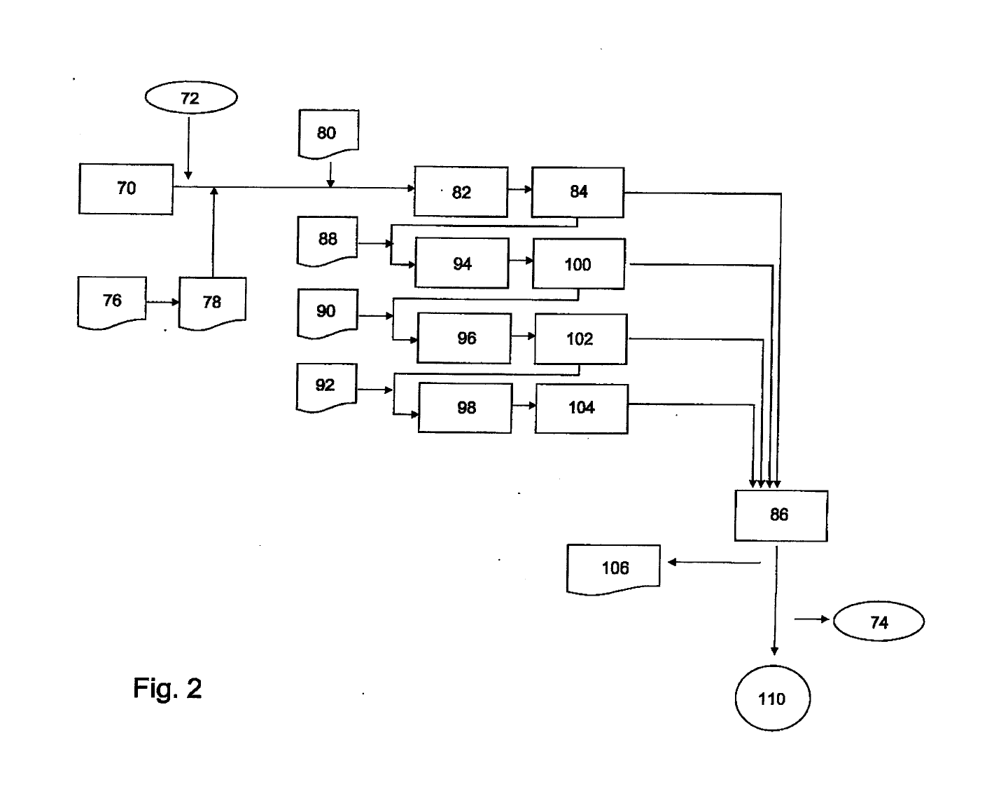

In FIG. 1, the basic course of a preferred embodiment of a method according to the invention for automated processing of a data source is illustrated, in particular, in the form of a conventional flowchart as it is used in computer programming. This representation begins usually with a block 10 that contains the title or name of the method, respectively, that is a component of a method combined of several methods. Such a method component is conventionally referred to as module. In the present case, the block 10 refers to the module “processing data source” that begins with a start statement 12 and, inasmuch as there is no branching to other modules or method components beforehand, ends with an end statement 14. The same holds true also for the method components illustrated in FIGS. 2, 3, 6, 8, 10, and 11 that are referred to in the following usually as “module” for short.

In the module illustrated in FIG. 1, first a data source 18 is read out of a so-called client folder 16 that contains all non-validated data sources and is acquired initially in a so-called data pool with regard to its existence (method step 20).

The data pool is the total of all information that is stored about a patient. The data pool contains preferably the following information elements:
ID data (identification data in regard to identifying the patient)
data regarding the data sources (for example, type, date of creation, result of evaluation) as a result of acquiring the data source in the date pool or as a result of an evaluation of the data source
data sources in stored form (scan, electronic data)
internal documents
health data (contents of the data sources) as a result of processing of the data source and of the contents documentation
structured summary (aggregate) of the health data
linking of the multi-dimensional structured referencing between elements of the data pool.

The client folder is a type of temporary collecting folder for all data sources whose evaluation, storage, processing, structured summary and multidimensional structured referencing have not yet been checked by validating the data pool.

In the client folder the incoming physical and electronic data sources for a patient are stored until the validation of the data pool is completed. The data sources are removed only for a brief period of time from the client folder when saving of the data sources is realized in the data pool of the patient. After saving, they are again integrated into the client folder and are available therein until completion of validation of the data pool. After completion of validation of the data pool the original documents in the client folder are returned to the sender or destroyed under consideration of the requirements of privacy. The client folder is then closed.

When a data source in the data pool has been acquired with regard to its existence, the medical and care-relevant information contents of the data source—for short referred to in the following as “health data”—are identified and also acquired in the data pool in the next method step 20.

The data sources can be rather different sources, for example:
written records in regard to examinations and treatments or dispensing of medication or medicinal products that have been compiled by physicians, hospitals, members of the medical care professions and medical assistance professions or drugstores,
formal documents such as vaccination cards, physician's letters, examination or surgery reports, prescriptions, medical opinions,
image data of examinations by means of imaging methods such as x-ray, ultrasound, magnetoresonance tomography, computer tomography, positron emission tomography, endoscopic examinations, photography e.g. of the skin,
product data such as data of implants, curative agents and auxiliary agents or other medical products with which the patient in question has been treated,
personal memories of the patient or his family without formal documentation.

The individual health data differ already when they are only superficially reviewed by a plurality of features, for example, with regard to:
the origin of information that can originate from the patient himself, his family, physicians, institutions of the health care system such as hospitals, health insurance companies, facilities of the public health care service, nursing personnel, members of the medical care profession and the medical assistance professions or drugstores,
the different quality of information, ranging from personal memories to formalized protocols of individual examinations to a summarized representation,
the age of the information,
the diagnostic and medical care significance,
the degree of accuracy,
the degree of its technical quality,
the media such as paper, film, electronic storage media.

Because of the different characteristics of each of these features for each piece of information in a data source that is relevant with regard to diagnostic or medical care considerations, i.e., the health data, it is not possible to determine the importance of the contents, validity, timeliness, and relevance of the respective information at first glance.

Since a systematic evaluation of the single individual health data has not been done previously in the medical care practice, in current medical care practice it cannot be determined ad hoc whether individual health data are accurate or false, whether the they conform to the requirements in regard to contents and technology, and whether they are to be considered or not for a decision-making process. This has the result that, because of unrecognized false or low-quality information, wrong decisions are made by the physicians or medical care personnel leading to consecutive damage being done to the patient. Also, often measures for obtaining information, for example, new examinations, are performed because a certain piece of information appears uncertain to the physician or nurse even though the information actually has high certainty. Unnecessary examinations however not only mean time loss and additional costs, they also cause unnecessary stress for the patient, for example, in the case of x-ray examinations.

The method according to the invention enables advantageously the removal of these disadvantages by a systematic structured checking process by means of which the importance of a data source is analyzed. This process, also referred to as evaluation of a data source, is illustrated in FIG. 2.

The module “evaluation data source” (70) with a start statement 72 and an end statement 74 begins in such a way that the health data 78 contained in a data source 76, i.e., all information that is not provided for identifying the patient but that describes his medical state, is retrieved. After retrieval these health data are checked with regard to certain criteria and evaluated based on a predetermined scale. For this purpose, first a checklist “origin of health data” (80) can be retrieved by means of which the method step “checking origin health data” (82) and the method step “rating origin health data” (84) is performed. The thus obtained results are then saved for determining a total result (method step 86). Additional checklists 88 (time interval between health event and generating a corresponding report), 90 (formal quality of the document), and 92 (contents quality of the document) are retrieved and, based on these checklists, in steps 94, 96, and 98 checks with regard to the time interval health event/report (94), formal quality of the document (96), and contents quality of the document (98) are realized. In the method steps 100, 102, 104, a rating with regard to the time interval health event/report (100), the formal quality of the document (102), and the contents quality of the document (104) is performed, respectively. The “ratings” are then intermediately saved for determining 86 the total results.

In this connection, the term “health event” refers to the state or the sequence of states that are the subject of a data source. The health event that is represented in a physician's letter can be, for example, the course of a treatment from the time of admission to the hospital to release. The health event represented in an x-ray image describes the state at the time of performing the x-ray examination and producing the x-ray image.

The results of the checks in regard to the individual criteria are then compiled in a so-called total rating (method step 86) that is comprised of sequentially arranged evaluations (ratings) in regard to the individual checks. The individual evaluations can be preferably expressed by simple grades or letters, for example, as known from the system of school grades or the evaluation of companies listed on stock exchange. In Tables 1 to 4 a possible rating system is shown where the “best grade” is the letter A, the “worst grade” is the letter E and criteria that cannot be evaluated are identified by numeral 0.

When using the evaluation system illustrated in Tables 1 through 4, the total rating of a physician's memorandum as a result of the individual ratings could be A-A-B-C, for example. The total rating of information provided by the patient in regard to an event that happened more than five years ago and for which no additional evidence is available, could D-E-E-E, for example. This total result leads to a documented evaluation of the data source (document 106 in FIG. 2). The module “evaluation data source” is then completed (74) and additional method steps, in particular, the module “multi-dimensional structured referencing” (110), could follow.

Such a method according to the invention enables thus advantageously that upon further processing of the health data evaluated in this way said health date can be immediately rated with regard to their relevance for a certain problem, automatically as well as manually.

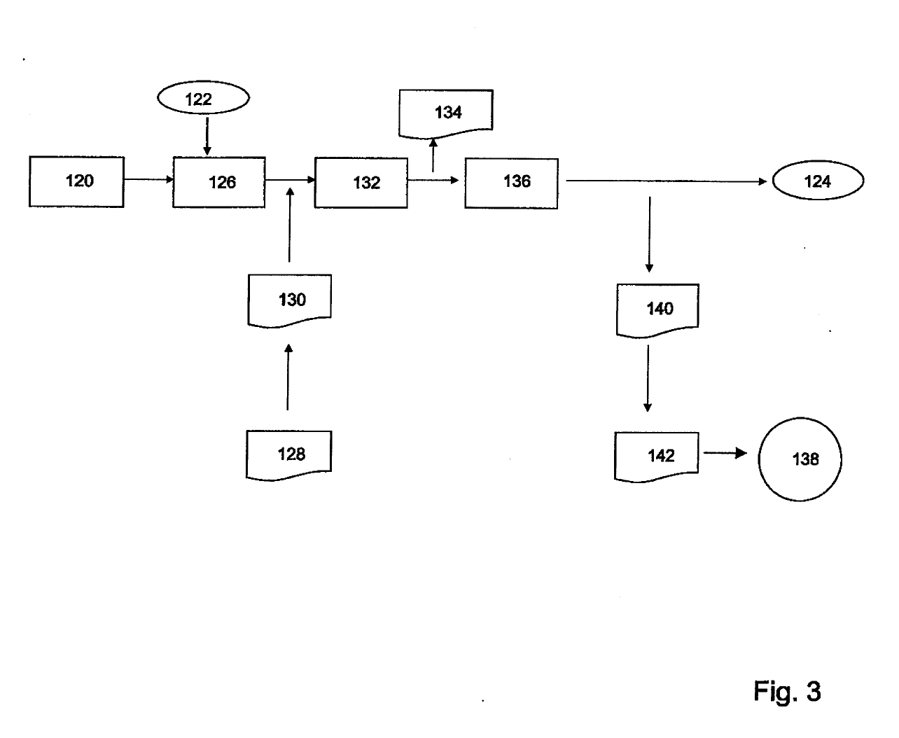

Before, subsequent to, or parallel to the method step of evaluating a data source, the data source (module 120) is saved in the data pool, as illustrated in FIG. 3. In this connection, the sequence illustrated in FIG. 3 is based on the premise that for a certain patient there is already a data pool available that can be opened (method step 126) and that contains the identification data of the patient and the previously acquired data in regard to the data source, for example. If there is no data pool for a patient available yet, it would first be created.

From the client folder 128 subsequently a data source 130 is retrieved and electronically saved in the data pool (method step 132); this leads to the data source being saved in the data pool (134). In a separate process (136), the data source is stored in the client folder until completion of authentication 138 within the database. For validation, the data source 140 is stored in the client folder 142 and transferred to the validation process. When validation is complete the client folder can be closed.

In FIG. 1 it is shown that after evaluation 24 and storage 26 of a data source (which leads to the health data 28 being saved in the data pool 30) a check of the contents of the data source based on predetermined parameters is realized, in particular, by means of the queries 32, 34, 36, and 38. Such a check can be performed, for example, under the aspect whether the information contained in the data source is complete or not in regard to a certain problem. For this purpose, in method step 32 it is checked whether the data source is complete.

When the data source is incomplete, research (method step 34) is required and the data source is then correspondingly marked for which purpose different processes are available. For example, it can be provided that when research is required automatically an acoustic and/or optical signal is generated and sent to a corresponding output device. However, the research request can also be started automatically, for which purpose, for example, the following procedure is carried out: depending on the type of information to be researched, either a database is automatically queried or an e-mail is automatically generated and sent to a researcher. In this way, the invention advantageously enables a person skilled in the art to select, depending on the application, an optimally matched procedure.

When processing a data source, for example, in the case of a data source of the type “internal document” in which, for example, personal memories of the patient or his family are recorded (internal documents secure all data about the patient that are not documented in external data sources, for example, information that the patient himself or the treating physicians make in a telephone conversation or hints that result from processing the external sources), it can happen that it is determined that another appointment is required, for example, because the provided information is not coherent (query 34—“appointment required?”). In this case, the data source can be marked accordingly such that automatically or manually an appointment with the patient or his family is to be scheduled (method step 42).

Certain data sources, for example, simple analyses of blood values and the like require a manual or automatic evaluation with regard to the meaning of the results (query 36—“evaluation data source required?”). These data sources can also be marked, in analogy to the already described way, such that still a manual or automatic evaluation of the respective data source is to be initiated or performed (method step 44).

In analogy it can be checked whether an evaluation of internal documents is required (query 38). When such a query is affirmed, the process 46 “evaluation internal documents” is carried out.

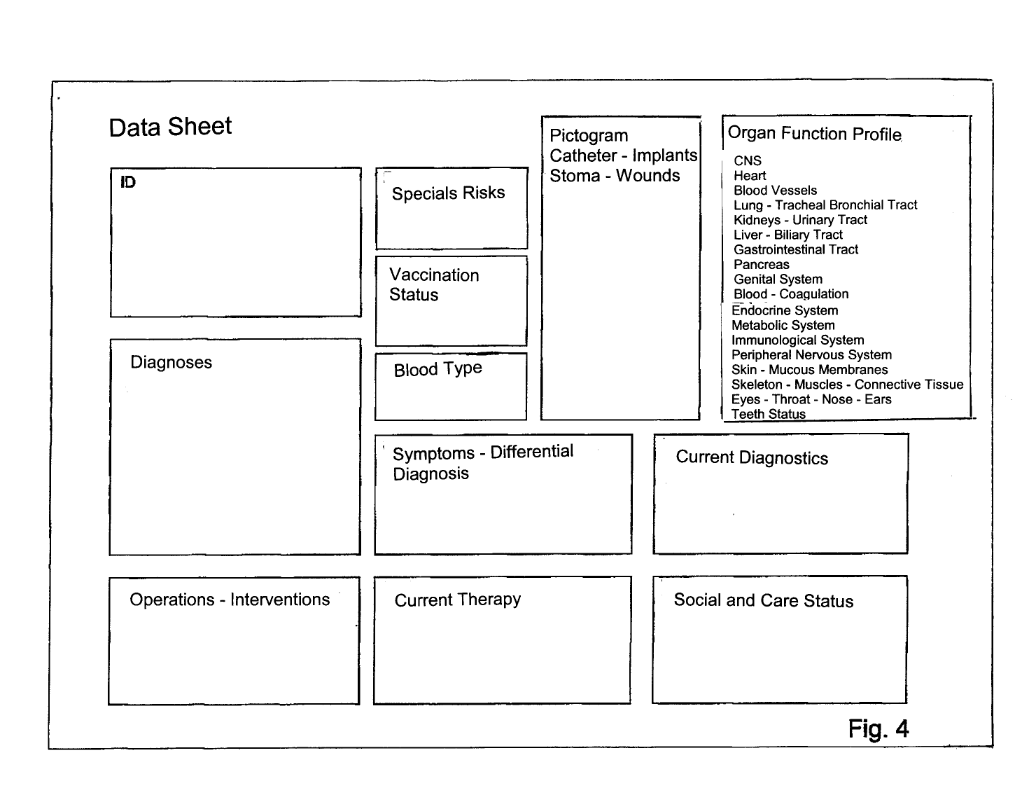

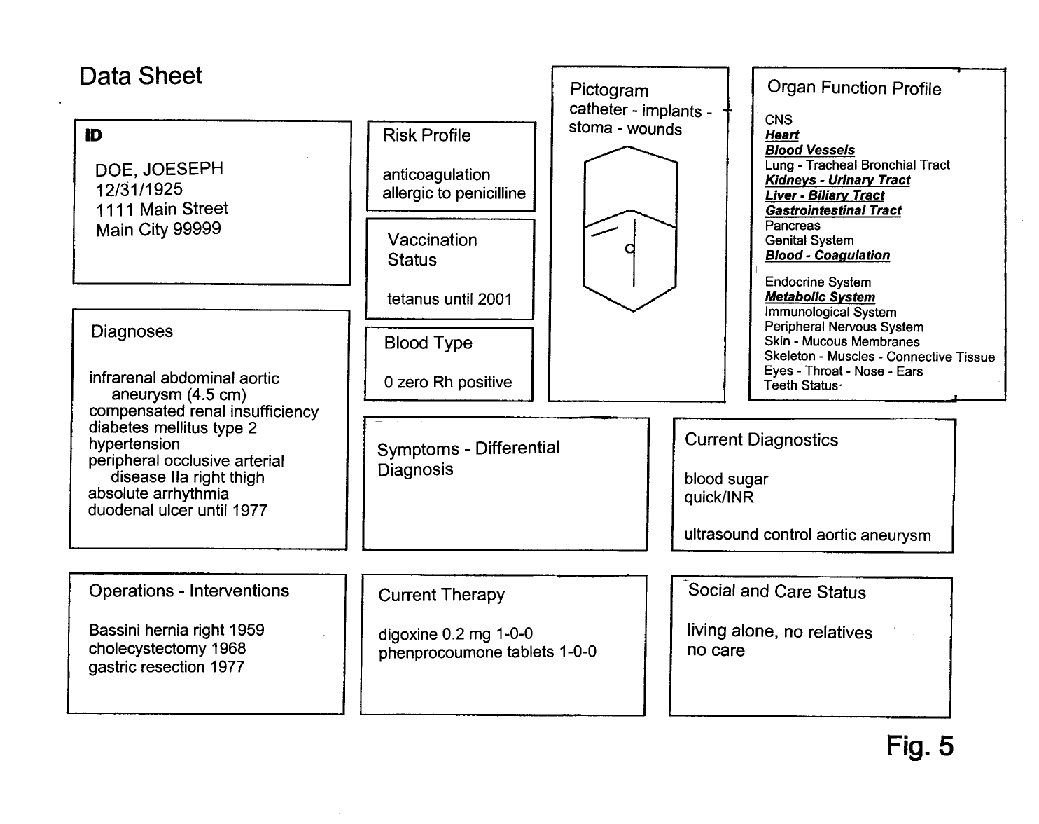

All sub-processes or processes 40, 42, 44, and 46 lead to the data source having to be processed, i.e., having to be treated in a separate method step 48. When the results of the queries 32, 34, 36, and 38 are negative, respectively, further processing 48 of the data source is not required and the health data contained in the data source can be automatically combined in a structured way (method step 50) in accordance with a predetermined schematic (predetermined boundary conditions) wherein the summary obtained in this way is referred to as an “aggregate”. In method step 52, said aggregate is transferred into a virtual so-called data sheet—to be described in the following in connection with FIGS. 4 and 5—and such a data sheet 54 with an aggregate is obtained.

The data contained in the virtual data sheet can also be referenced (module 56—“multidimensional structured referencing”), and the data pool that is derived from all data sources and the information contained in the data sources can be validated (module 58—“validating data pool”).

In FIG. 4 and FIG. 5 such a virtual data sheet is illustrated wherein in FIG. 4 a possible basic structure of the virtual data sheet and in FIG. 5 a virtual data sheet with exemplary data is illustrated.

The virtual data sheets can be made available to the physician, the nurse or family or other health care professions and health assistance professions in different forms and facilitate their daily work significantly. It is especially advantageous when the virtual data sheets are stored and maintained at a central location. When a patient visits different physicians, they can transmit actual information to the central location where the virtual data sheet is updated and made available for retrieval by authorized persons, for example, other physicians. In this way, in the case of caring for an emergency patient or an accident victim in a hospital, it is possible to have available immediately all required information for treating the patient.

The data sheet illustrated in FIG. 4 and FIG. 5 contains the following information blocks:
identification data of the patient,
diagnoses,
blood type,
vaccination status,
performed, actual, and planned therapies,
social and care status,
special risks,
organ function profile,
unsolved symptoms, findings, differential diagnoses, as well as
a pictogram for showing e.g. the position of surgery scars and the like.

Data sheets having such a configuration have found wide acceptance but it is of course possible to include additional or other information blocks on the data sheet and/or to arrange the information blocks differently. Standardization of the data sheet however has the advantage that the user of the data sheet, once they have become familiar with the structure of the data sheet, can retrieve very quickly information from the data sheet because information of a certain type is always arranged at the same location on the data sheet.

In the example illustrated in FIG. 5, the pictogram shows the patient's trunk that shows two surgery scars whose approximate length is indicated by the relative position to the navel and the two lowermost ribs that are also indicated in the pictogram.

An especially advantageous embodiment of the method according to the invention provides that the health information available for a patient is referenced, i.e., linked with one another in such a way that the interrelations become visible as needed and correlated information can be retrieved in a simple way, for example, by means of the virtual data sheet.

The virtual data sheet can show not only the most important basic information for the physician or nurse but can advantageously also serve as a retrieval mask for the data pool upon which the data sheet is based. For this purpose, the individual elements in the data sheet can be configured as hyperlinks so that by clicking on one element of the data sheet represented on a screen further information in regard to the selected element can be retrieved, i.e., information in regard to the heart or the central nervous system.

In the past, in daily practice the health data that are the basis for medical and care-giving decisions when treating a patient are not correlated with one another and not linked with one another even though they belong together with regard to contents. For this reason, comparative evaluations between prior and current states, for example, of x-ray images or findings reports, generally cannot be performed easily.

When health data and documents are to be correlated with one another in accordance with medical/care-giving criteria, a complex search for the required documentation in a plurality of institutions is required currently. This is followed by time-consuming file studies, review of a plurality of image and text documents, and contents correlation in the context of an evaluation.

Even when in the patient file all documents are physically combined within a certain treatment interval because of the fact that a patient has been treated for a long time by one and the same physician, a fast linking of the individual information contained in the patient file, as it is needed, for example, in the case of emergency treatments, cannot be carried out easily because the patient file is only chronologically structured but not structured with regard to contents.

In the context of different scientific projects it has been attempted to combine data sources in so-called electronic patient files. These patient files however solve only the problem of distributed archiving because they can combine the data sources of different institutions and different time periods. They do not solve the problem of contents-related linking of health data and data sources that is urgently needed for accessing relevant health data, for example, in the case of patients with special health risks or in emergency situations.

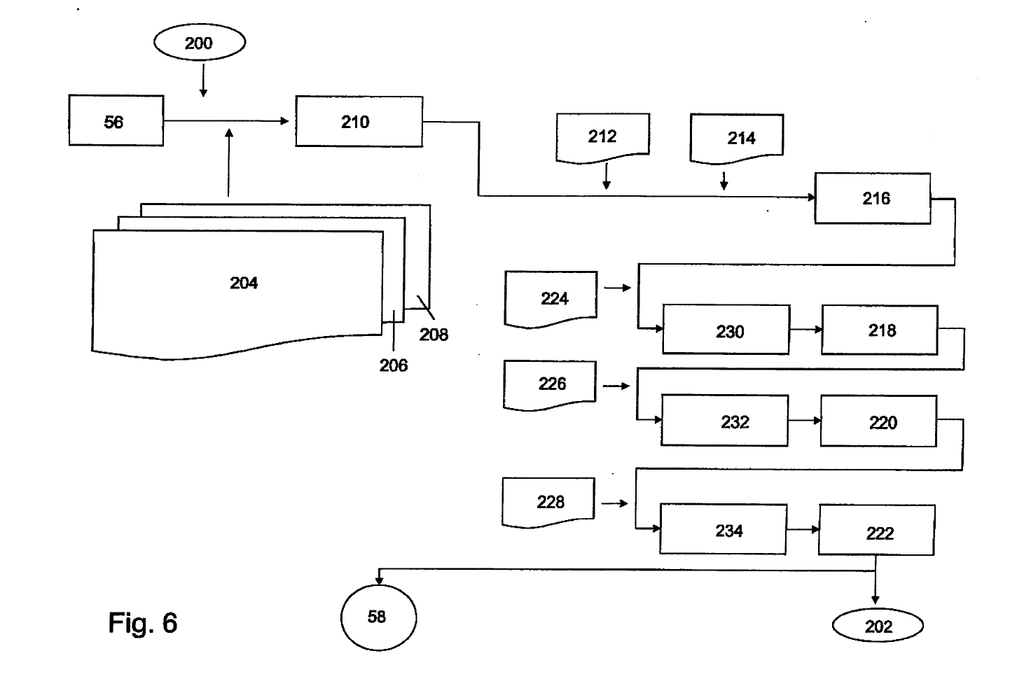

The invention now proposes to combine correlated health data and the data sources that are the basis for the health data according to predetermined medical or care-giving criteria, as illustrated in FIG. 6.

FIG. 6 shows the module “multi-dimensional structured referencing” that in FIG. 1 is referenced with reference numeral 56 and that contains for realization by a computer program a start statement 200 and an end statement 202. The module 58 (validation data pool) is connected downstream of the module 56. For multi-dimensional structured referencing, first the already present information elements, of which in FIG. 6 three are shown as an example and referenced by reference numerals 204, 206, and 208, are read out and subsequently analyzed and linked with one another in the method step 210 taking into account the evaluation of the individual data sources that has been carried out before; for this purpose, the result of evaluation of the data sources 212 and the individual data sources in saved form 214, containing the individual health data, are retrieved.

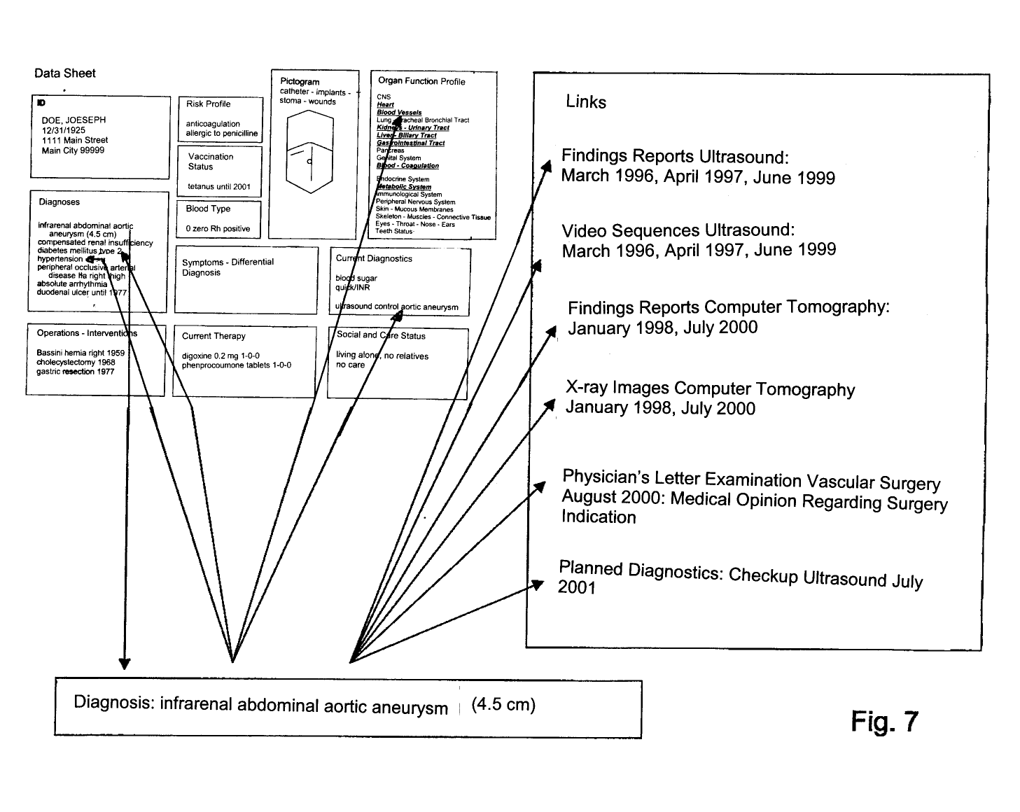

The technical configuration of linking is realized in method step 216. For example, linking of the information “diagnosis XY” with those data sources that are the basis of this diagnosis and provide evidence for the diagnosis. This is illustrated in FIG. 7 in an exemplary way.

In further method steps 218, 220, and 222, additional links are created that provide the connection between diagnosis, the correlated subsequent examinations, and the additional planned measures. Moreover, links between certain aggregated health data, for example, diagnosis “gallbladder stone ailment” and the operation “gallbladder removal” can be made. Also, links between diagnoses and the correlated organ system can be provided that enable momentarily the accumulation of multiple stress situations for certain organ systems. For this purpose, the structured summaries 224 (aggregates) of the health data, the contents of the data sources, i.e., the health data 226, as well as the data sources in their stored form 228 are retrieved. In method steps 230, 232, and 234 a contents definition of links to other information elements is realized.

As a last method step, in the preferred embodiment the so-called validation of the data pool (module 58) is provided which follows referencing. In accordance with ISO 9000 validation means making available an objective proof that the requirements for an intended use or a specific intended application have been fulfilled.

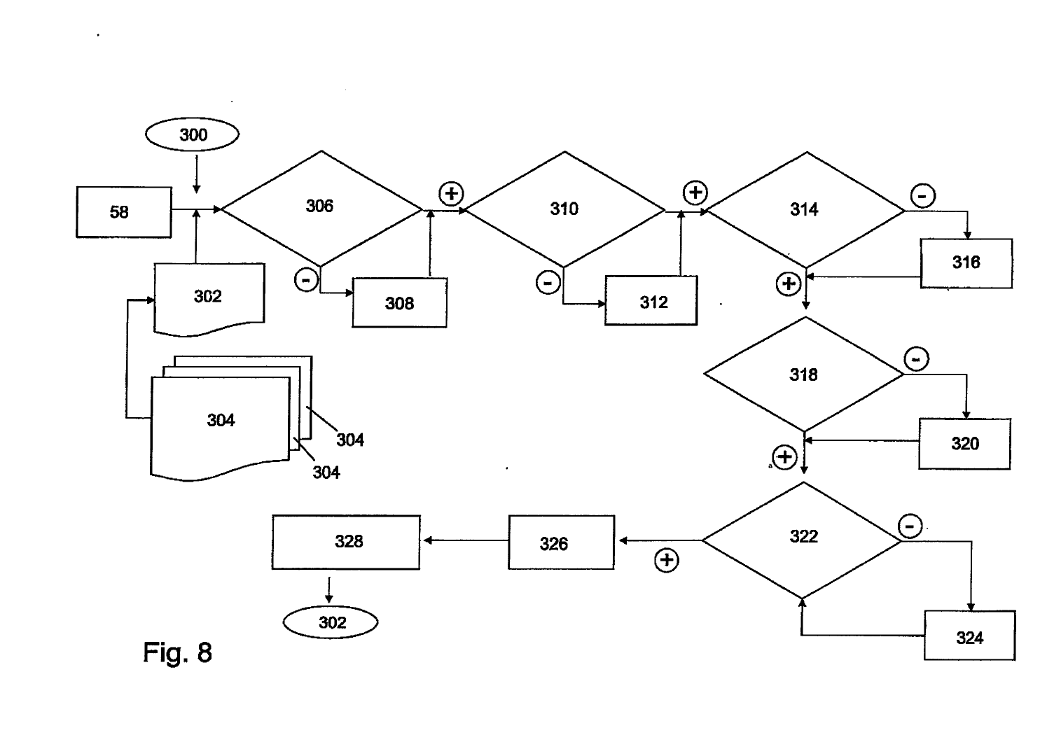

FIG. 8 shows such module 58 in the conventional illustration between a start statement 300 and an end statement 302. As the basis of the test method the patient folder 302 is read out that contains all non-validated data sources 304, i.e., all data sources that have not yet passed the process “validation data pool”.

In the method step 306 it is then checked whether the identification data of the data sources in the patient folder coincide with the identification data in the data pool; if this is not the case a correction of the data that do not coincide is carried out (method step 308).

Subsequently, in the method step 310 the data in regard to the data sources in the data pool are tested in regard to whether for the data corresponding data sources exist in the patient folder. If this is not the case, in the method step 312 a correction of non-coinciding data is carried out.

Subsequently, in the method step 314 the contents of the data sources in the client folder are compered with the contents data, i.e., the health data in the data pool are compared. When errors are found, a correction of erroneous data is carried out in the method step 316.

In an additional method step 318 a validation is provided whether the contents of the data sources in the client folder coincide with the structured summary (aggregate) of the contents of the data sheet. If this is not the case, a correction is done in method step 320.

Finally, in the method step 322 the correctness of the contents of the links of referencing within the data pool is tested. Possible errors are corrected in method step 324.

When in this test no errors have been found, in the method step 326 the data pool and in the method step 328 the structured summary of the data pool are released in the form of a data sheet.

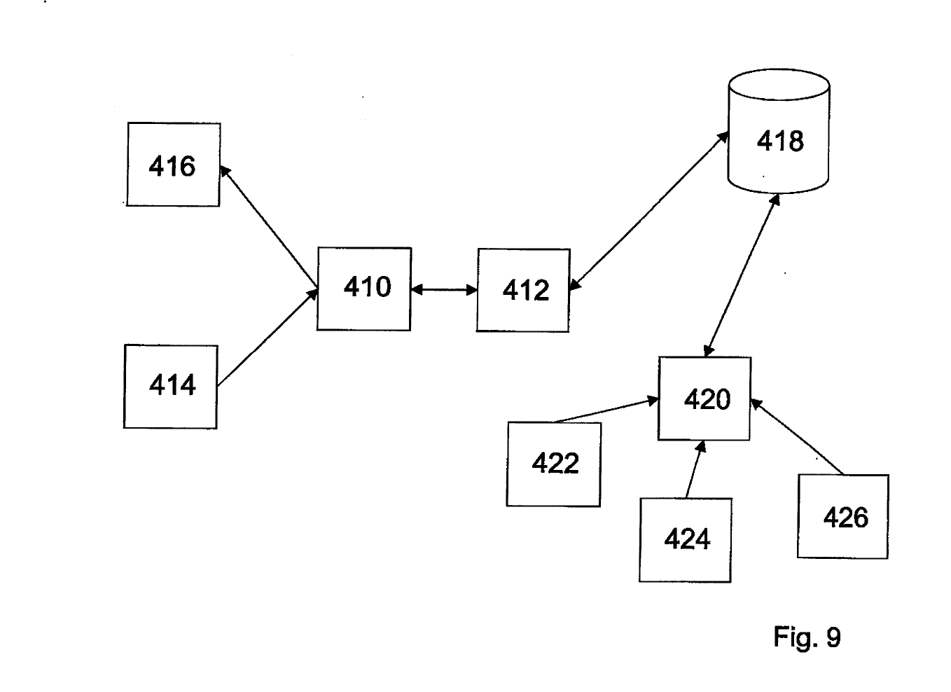

In FIG. 9 a purely schematic illustration of a possible arrangement for performing the method according to the invention is illustrated in which a first data processing unit 410 and a second data processing unit 412 are connected to one another wireless or by wire-bound lines, in particular, by means of a data net such as the Internet for the purpose of data exchange.

The first data processing unit 410, for example, is accessible by a physician or a hospital and is provided with at least one input unit 414 in the form of a keyboard and/or a mouse and at least one output unit 416, in particular, in the form of a screen.

The second data processing unit 412 manages and monitors the access to a data base 418 in which the data pools of the patients are stored (generally, each patient has correlated therewith a data pool and several data pools form a database).

In this connection, it is understood that neither the database 418 nor the second data processing unit 412 must be a single component but that instead the databases can be distributed onto many different storage media and the access and administration can be controlled by several data processing units. This not only serves for managing, as needed, many access requests at the same time but also for ensuring that access is still possible when one of the data processing units fails. For reasons of simplification, in the purely schematic illustration of FIG. 9 only one second data processing unit 412 and one database 418 are illustrated.

The data in the database can be maintained by the second data processing unit 412. For security reasons, in practice the technical solution will be usually such that the database can be maintained only by a separate third data processing unit 420. This third data processing unit has several input devices 422, 424, and 426 that enable, for example, scanning of documents that are available in paper form, reading of documents that are present on different storage media, and manually inputting information that is not automatically acquired.

The illustrated arrangement operates as follows. First by means of the third data processing unit 420 a data pool and a client file are generated for a patient, wherein in the client file all non-validated data sources, i.e., such data sources that have not yet passed the process of validation of the data pool, are entered. Subsequently, the individual data sources are processed in the above described way so that finally a virtual data sheet and a validated data pool can be saved in the database 418. Preferably, the data are also referenced so that in the above described way links between the individual information can be generated.

When a physician needs the actual patient file, a connection is made by means of the data lines between the data processing unit 410 that the physician can access and the second data processing unit 412 so that, as is conventional, first proper authorization of the requesting data processing unit 410 in regard to access to certain patient files is checked. When this security check has been completed, the second data processing unit 412 reads out the requested data from the database 418 and transmits the data to the first data processing unit 410 so that the data can be output on the output device 416.

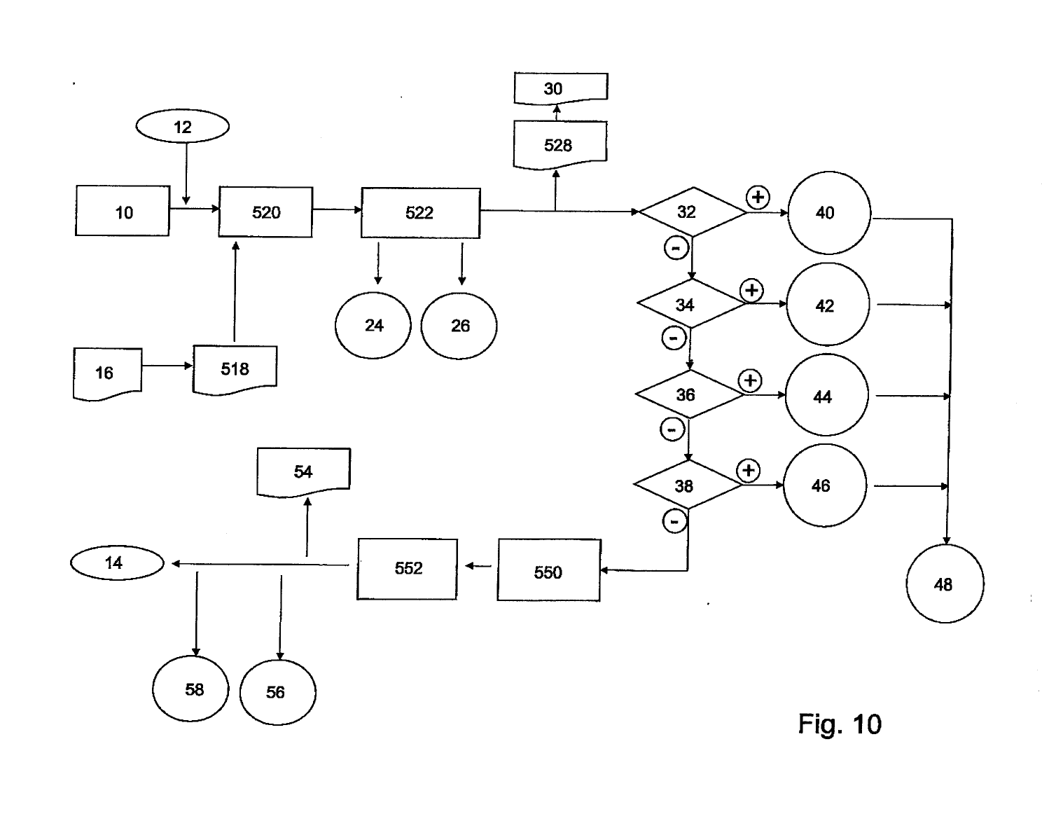

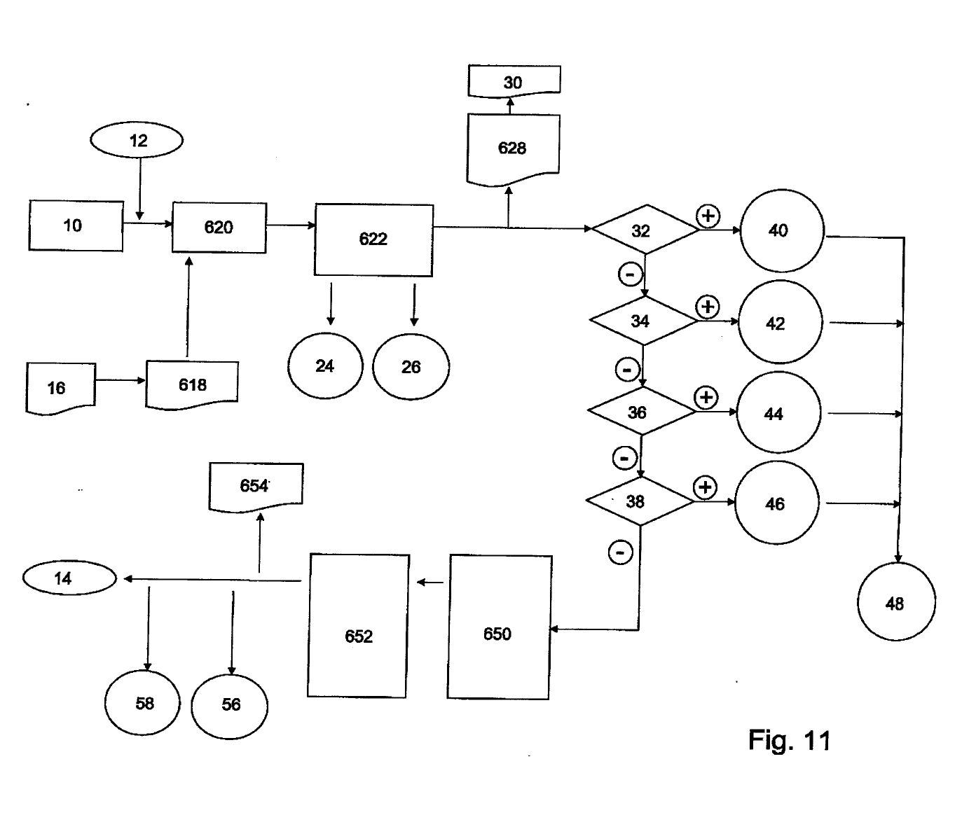

In FIG. 10 and FIG. 11, in an exemplary fashion the course of the method step “processing data source” according to FIG. 1 is illustrated wherein the data source to be processed in the example of FIG. 10 is an x-ray image 518 and in the example of FIG. 11 is a physician's letter 618. The method steps, for example, evaluation data source (method step 24) or saving data source (method step 26) have already been explained supra in detail in connection with FIG. 1 so that reference is being had to the description above.

In the method step 520, first the x-ray image in the data pool is detected. In the method step 522, the recognized data source is acquired with regard to its contents, i.e., in regard to its importance, in the database. For example, in the method step 522 it could be acquired that pneumonia is present in the left lung. The health data 528 contain therefore the entry “pneumonia left lung”.

In the above described way, in the steps 32, 34, 36, and 38 it is checked whether processing of the data source is required. If this is not the case, finally the structured summary (method step 550) with the diagnosis “pneumonia left lung” is prepared. This diagnosis is transferred in method step 552 into the data sheet.

In the example of FIG. 11, the data source 618 is a physician's letter. In the method steps 620 it is first acquired in the data pool. In the method step 662 the contents of the letter is then acquired in the data pool wherein this contents, for example, can be “pneumonia left lung, antibiotic treatment; cured”. Accordingly, the health data 628 contain the entry “pneumonia left lung; antibiotic treatment; cured”.

In the above described way, in method steps 32, 34, 36 and 38 it is checked whether further processing of the data source is required. If this is not the case, in the method step 650 finally the structured summary is provided that contains the following: “diagnosis: pneumonia left lung; therapy: antibiotic treatment; course: cured”.

This structured summary (the aggregate) is then transferred in method step 652 into the data sheets 654.

In physician's letters but also in a series of other data sources, there are often so-called diagnosis codes that serve to code certain diagnosis in such a way that they can be recognized and further processed more easily byway of currently used data processing systems. For example, in Germany a coding system has been introduced in which about 25,000 different diagnoses each have correlated therewith a code comprised of letters and numerals. At present, physicians in hospitals who use such a system must convert the respective diagnosis after a consultation by means of complex tables into a corresponding diagnosis code; this can easily lead to input errors.

The invention can be performed preferably such that in those data sources in which a diagnosis code is contained the diagnosis code is automatically checked with regard to correctness and that, when errors appear or at least very rare diagnoses are found during this check, a corresponding signal is generated which has the result that the corresponding data source of the corresponding diagnosis must be checked again and affirmed.

If the diagnosis code contained in the data source, for example, stands for the diagnosis “leprosy” and the patient is an otherwise healthy Western European, the probability is very high that the diagnosis code in question is a wrong input. The method according to the invention can thus automatically cause the diagnosis to be affirmed or corrected.

Preferably, a database, in particular, an autodidactic database can be provided that contains typical diagnosis code errors, for example, typing errors that occur frequently, for example, K29.2 instead of K92.2. When such a database is provided, the method can be performed such that when incoherent data occur a corrective proposal is made automatically and displayed on a corresponding output, for example, a screen (this is known e.g. from word processing programs in regard to spell checking).

Of course, in those cases where the known coding system is not to be eliminated or cannot be eliminated because of laws and regulations, the method can be performed such that a certain diagnosis is automatically converted into the corresponding diagnosis code.

In the context of the present invention numerous deviations and further developments are possible which relate, for example, to the number and selection of criteria when evaluating a data source or the combination of the health data in the virtual data sheet. Also, it should be noted that the invention implies a new method of doing business, i.e., the economically especially interesting operation of a medical database.

Clients of such a database can be, on the one hand, physicians and hospitals for whom the access to continuously maintained medical information of a patient, which information is also acquired by other physicians and hospitals, is very interesting with regards to proper care but also for economical reasons and reasons of national economics (avoiding duplicate examinations).

Clients of such a database can also be the individual patients themselves who have an interest in an especially excellent medical care or have an increased disease risk and who want to make sure that also in emergency situations a physician who is not familiar with the anamnesis of the patient can have immediately access to the complete patient file. This method is therefore expressly encompassed in the present invention and is also claimed in those countries where national law allows for this.

TABLE 1
Origin of Health Information

| Criterion | Contents | Rating |
|---|---|---|
| Origin of | Documents | A |
| Health | Foreign Information from Physicians without | B |
| Information | Evidence | |
| | Foreign Information from Other Heath Care | C |
| | Professions without Evidence | |
| | Own Information from Patient without Evidence | D |
| | Foreign Information from Family without | E |
| | Evidence | |
| | Origin Unknown | 0 |

TABLE 2
Time Interval between Health Event and Report

| Criterion | Contents | Rating |
|---|---|---|
| Time Interval | No Interval | A |
| between | up to 6 Months | B |
| Health Event | 6 to 12 Months | C |
| and Report | 1 to 5 Years | D |
| | more than 5 Years | E |
| | Interval Cannot Be Determined, No Data | 0 |

TABLE 3
Formal Quality of the Document

| | Contents | | | |
|---|---|---|---|---|
| Criterion | Partial Criterion | | Points | Rating |
| Formal | (1) | Complete Data in Regard to | 2 | |
| Quality | | ID of Patient | | |
| of | (2) | Complete Date | 2 | |
| Documents | (3) | Complete Data in Regard to | 2 | |
| | | Author ID | | |
| | (4) | Signature of Author | 2 | |
| | (5a) | Preliminary Document | 1 | |
| | (5b) | Final Document | 2 | |
| | (6a) | Brief Report | 1 | |
| | (6b) | Detailed Report | 2 | |
| | (7) | None of the Criteria | 0 | |
| | | (1)-(6b) fulfilled | | |

| Sum of Evaluation of the Partial Criteria (1)-(7) | |
|---|---|
| (Points) | Rating |
| 12 | A |
| 9-11 | B |
| 7-8 | C |
| 4-6 | D |
| 1-3 | E |
| 0 | 0 |

TABLE 4
Contents Quality of the Document

| | Contents | | | |
|---|---|---|---|---|
| Criterion | Partial Criterion | | Points | Rating |
| Contents | (1) | Examination - Intervention | | |
| Quality | (1a) | Anamnesis | 1 | |
| of the | (1b) | Indication Data | 1 | |
| Document | (1c) | Findings Data | 1 | |
| | (1d) | Data re Intervention or | 1 | |
| | | Surgical Measures | | |
| | (1e) | Course Data (1) | 1 | |
| | (2) | Summary | | |
| | (2a) | Diagnosis Data | 1 | |
| | (2b) | Exclusion Data | 1 | |
| | (2c) | Suspicion Data | 1 | |
| | (3) | Coding | | |
| | (3a) | ICD (international | 1 | |
| | | classification of diseases) | | |
| | (3d) | OPS (operation and procedure | 1 | |
| | | system) | | |
| | (3c) | Use of Other Classification | 1 | |
| | (4) | Additional Measures | | |
| | (4a) | Recommendations | 1 | |
| | (4b) | Documented Transfer | 1 | |
| | (5) | None of the Partial Criteria | 0 | |
| | | (1a)-(4b) fulfilled | | |

| Sum of Evaluation of Partial Criteria (1a)-(5) | |
|---|---|
| (Points) | Rating |
| 10 and more Points | A |
| 8-9 Points | B |
| 6-7 Points | C |
| 3-5 Points | D |
| 1-2 Points | E |
| 0 Points | 0 |

What is claimed is:

1-10. (canceled)

11. A method for automatically processing and evaluating medical data for the purpose of determining and providing diagnostic and/or medical-care relevant information based on a plurality of available data sources, the method comprising the steps of:
acquiring all data sources in a common data pool;
evaluating each data source based on predefined criteria;
saving results of the step of evaluating in correlation to the data sources, respectively;
referencing data contained in the data pool such that elements of the data pool that are correlated with regard to contents are linked and displayable automatically as being correlated;
producing, based on the data sources in accordance with predetermined boundary conditions, a structured summary in the form of a virtual data sheet.

12. The method according to claim 11, wherein the predefined criteria based on which each data source is evaluated comprises at least one criterion selected from the group of criteria consisting of: origin of a data source, quality of a data source, age of a data source, formal quality of a data source, and contents quality of a data source.

13. The method according to claim 11, further comprising the step of combining information in blocks on the virtual data sheet, wherein the blocks contain information in regard to at least one aspect of the group of aspects consisting of: identity of a patient, special risks, vaccination status, blood type, diagnoses, surgeries/interventions, current therapies, symptoms/differential diagnosis, current diagnostics, catheter/implants, stoma/wounds, organ function profile central nervous system, organ function profile heart, organ function profile blood vessels, organ function profile lungs/tracheal bronchial tract, organ function profile kidneys/urinary tract, organ function profile liver/biliary tract, organ function profile gastrointestinal tract, organ function profile pancreas, organ function profile genital system, organ function profile blood/coagulation, organ function profile endocrine system, organ function profile metabolism, organ function profile immunological system, organ function peripheral nervous system, organ function profile skin/mucous membranes, organ function profile skeleton/muscles/connective tissue, organ function profile eyes/throat/nose/ears, organ function profile teeth status, social status, and care status.

14. The method according to claim 11, wherein the predetermined boundary conditions take into account the results of the step of evaluating the data sources.

15. The method according to claim 11, wherein the data pool is validated by being checked based on predetermined criteria in regard to whether the data contained in the data pool are correct.

16. The method according to claim 11, further comprising the steps of displaying the virtual data sheet on a screen and configuring the virtual data sheet as a hypertext document such that by clicking on an element of the data sheet additional elements of the data sheet or of the data pool that are linked with said element are retrieved.

17. The method according to claim 16, further comprising the step of configuring the virtual data sheet such that by clicking on one of the elements of the data sheet all of the additional elements linked therewith and indicated within the data sheet are visually highlighted so that immediately all correlated elements are recognizable and by clicking again or by double-clicking one of the elements of the data sheet a list opens in which list at least the data sources upon which said one of the elements of the data sheet is based are retrievably combined.

18. The method according to claim 11, further comprising the step of automatically checking correctness of a diagnosis code of the data sources when the data sources contain a diagnosis code.

19. The method according to claim 18, further comprising the step of querying a preferably autodidactic database containing typical diagnosis code errors when the step of automatically checking correctness indicates an error.

20. An arrangement for automatic processing and evaluating medical data for the purpose of determining diagnostic and/or medical-care relevant health data based on a variety of available data sources, the arrangement comprising a computer system comprising several components and configured for performing the method according to claim 11.
